(19) United States
(12) Patent Application Publication
(10) Pub. No.: US 2026/0174791 A1
(43) Pub. Date: Jun. 25, 2026

(54) MACROPHAGE MIGRATION INHIBITORY FACTOR (MIF) TARGETING FOR THE TREATMENT OF NON-SMALL CELL LUNG CANCER

(71) Applicant: Board of Regents, The University of Texas System, Austin, TX (US)

(72) Inventors: John Victor Heymach, Austin, TX (US); Ana Galan-Cobo, Austin, TX (US)

(21) Appl. No.: 19/127,681

(22) PCT Filed: Nov. 1, 2023
(86) PCT No.: PCT/US2023/036572

§ 371 Date: May 6, 2025

Related U.S. Application Data
(60) Provisional application No. 63/423,444, filed on Nov. 7, 2022.

(51) Int. Cl. A61K 31/713; A61K 45/06

(52) U.S. Cl. CPC A61K 31/713; A61K 45/06

(57) ABSTRACT

Provided herein are methods of reducing immune checkpoint therapy resistance, increasing immune checkpoint therapy sensitivity and/or increasing anti-tumor activity in subjects with cancer, methods of treating non-small cell lung cancer (NSCLC), and methods of identifying a subject as a responder to a macrophage migration inhibitory factor (MIF) targeting agent therapy. The methods include identifying alteration in the KEAP1/NRF2 pathway and administering MIF targeting agent therapy. Illustrative immune checkpoint therapies include PD-1 inhibitors and/or PD-L1 inhibitors.

### CROSS-REFERENCE TO RELATED APPLICATIONS

This application claims benefit of priority under 35 U.S.C. § 119(e) of U.S. Provisional Application No. 63/423,444, filed Nov. 7, 2022. The disclosure of the prior application is considered part of and is herein incorporated by reference in the disclosure of this application in its entirety.

### BACKGROUND OF THE INVENTION

### Field of the Invention

The present invention relates generally to methods of increasing cancer cell sensitivity and more specifically to the identification of KEAP1/NRF2 pathway alterations as a biomarker of macrophage migration inhibitory factor (MIF)-responsive tumors.

### Background Information

In lung adenocarcinoma (LUAD), KEAP1 is the third most common tumor suppressor and loss-of-function mutations in KEAP1 commonly co-occur with STK11/LKB1 and KRAS mutations. KEAP1 protein regulates the degradation of the antioxidant transcription factor NRF2. The role of STK11/LKB1 mutations in immunotherapy resistance has been characterized, however the mechanistic understanding of KEAP1 deficiency in shaping LUAD phenotype and therapy response is still very limited. Recent clinical data has been reported suggesting that mutations in STK11/LKB1 and KEAP1 are strongly associated with immune checkpoint blockade resistance in LUAD, particularly those with KRAS mutations. Nevertheless, the biology of KEAP1-deficient tumors and the immune suppression mechanisms are to be characterized.

Alterations in the KEAP1/NRF2 (K/N) pathway define a subgroup of non-small cell lung cancers associated to poor prognosis and reduced responsiveness to chemotherapy and immunotherapy. These alterations include mutations in KEAP1 or NFE2L2/NRF2, genomic loss of KEAP1, reduced KEAP1 protein, increased NRF2 protein, or mRNA expression signatures for the pathway.

Lung cancer is one of the most malignant tumors with the fastest growth in incidence and mortality rates, which poses the greatest threat to the health and lives of human beings. According to histopathology, lung cancer is mainly divided into two categories, small cell lung cancer (SCLC) and non-small cell lung cancer (NSCLC). Specifically, NSCLC accounts for about 80% of all lung cancers. Due to the slow growth rate of cancer cells and the late spread of metastasis, once NSCLC is clinically diagnosed, it is usually in its advanced stage; therefore, the 5-year survival rate of NSCLC patients is extremely low. In tumor diseases, the internal and external environment in which tumor cells are located has an important influence on the occurrence, growth, and metastasis of tumors. Heterogeneous tumor cells and non-tumor cells coexist in tumors, and the living environment provided by non-tumor cells for protooncogenes is called tumor microenvironment. In the tumor microenvironment, tumor cells can change and maintain their own survival and development conditions through autocrine and paracrine, thereby assisting the growth and development of tumor. Studies have found that the macrophage migration inhibitory factor (MIF) can assist tumor microenvironment and participate in tumor development. MIF is a protein molecule with multiple potencies that is constitutively expressed in a variety of immune and non-immune cells. High expression levels of MIF are observed in a variety of cancers. MIF plays an important role in the angiogenesis of tumor diseases, and MIF knock down significantly inhibits the development of lung adenocarcinoma suggesting that MIF plays an important role in tumor progression, but how MIF participates in the development of NSCLC has not been fully described.

### SUMMARY OF THE INVENTION

The present invention is based on the seminal discovery that an alteration in the KEAP1/NRF2 pathway in a cancer cell is associated with increased levels and/or secretion of macrophage migration inhibitory factor (MIF), which can be targeted by MIF targeting agents to sensitize cancer cells to therapy, e.g., immune checkpoint inhibitors.

In one embodiment, the present invention provides a method of treating non-small cell lung cancer (NSCLC) in a subject including: a) identifying an alteration in a KEAP1/NRF2 pathway in a cancer cell in the subject, and b) administering to the subject identified with a KEAP1/NFR2 pathway alteration, an anti-macrophage migration inhibitory factor (MIF) agent, thereby treating NSCLC in the subject.

In one aspect, identifying an alteration in a KEAP1/NFR2 pathway includes detecting a KEAP1 gene mutation, NFE2L2 (NRF2) gene mutation, genomic loss of KEAP1, reduced KEAP1 protein expression, increased NRF2 protein expression, and/or protein and/or gene expression modification in a gene or protein in the KEAP1/NRF2 pathway in the cancer cell. In another aspect, the cancer cell further includes a KRAS mutation and/or a STK11/LKB1 alteration. In one aspect, the MIF targeting agent is selected from the group consisting of an anti-MIF antibody, anti-MIF isoform antibody, anti-oxidized MIF antibody, anti-MIF receptor antibody, MIF receptor inhibitor, CD74 inhibitor, MIF blocking peptide, CAR-T cell, anti-MIF antibody drug conjugate and anti-MIF small molecule inhibitor. In another aspect, the method further includes administering to the subject a further therapeutic treatment. In some aspects, the therapeutic treatment is selected from the group consisting of immune checkpoint inhibitor therapy, chemotherapy and radiotherapy. In various aspects, the immune checkpoint inhibitor therapy includes a PD-1 inhibitor and/or a PD-L1 inhibitor. In many aspects, the PD-1 inhibitor and/or a PD-L1 inhibitor are selected from the group consisting of nivolumab, pembrolizumab, atezolizumab, avelumab, durvalumab, cemiplimab, and dostarlimab. In one aspect, treating NSCLC includes inhibiting NSCLC cell proliferation, reducing NSCLC tumor growth and/or increasing NSCLC patient survival.

In another embodiment, the invention provides a method of reducing immune checkpoint therapy resistance, increasing immune checkpoint therapy sensitivity and/or increasing anti-tumor activity in a subject having cancer including administering to the subject an anti-macrophage migration inhibitory factor (MIF) agent, wherein the subject's cancer cells have a KEAP1/NRF2 alteration, thereby reducing immune checkpoint therapy resistance, increasing immune checkpoint therapy sensitivity and/or increasing anti-tumor activity in the subject.

In one aspect, the subject has non-small cell lung cancer (NSCLC). In another aspect, reducing immune checkpoint therapy resistance and/or increasing immune checkpoint therapy sensitivity includes increasing cancer cell PD-L1 expression, decreasing MIF level and/or MIF secretion, and/or increasing anti-tumor activity. In one aspect, the immune checkpoint therapy includes a PD-1 inhibitor and/or a PD-L1 inhibitor. In some aspects, the PD-1 inhibitor and/or a PD-L1 inhibitor are selected from the group consisting of nivolumab, pembrolizumab, atezolizumab, avelumab, durvalumab, cemiplimab, and dostarlimab. In another aspect, increasing anti-tumor activity includes reversing MIF-induced cancer cell immunosuppressed phenotype, increasing cancer cell sensitivity to immune checkpoint blockade, inhibiting tumor-associated immune evasion, increasing tumor immune infiltration and/or inhibiting pro-tumorigenic M2 macrophage polarization. In one aspect, the MIF targeting agent is selected from the group consisting of anti-MIF antibody, anti-MIF isoform antibody, anti-oxidized MIF antibody, anti-MIF receptor antibody, MIF receptor inhibitor, CD74 inhibitor, MIF blocking peptide, CAR-T cell, anti-MIF antibody drug conjugate and anti-MIF small molecule inhibitor. In another aspect, the method further includes administering to the subject a further therapeutic treatment. In some aspects, the treatment is selected from the group consisting of immune checkpoint inhibitor therapy, chemotherapy and radiotherapy. In one aspect, the alteration in the KEAP1/NRF2 pathway is selected from the group consisting of a KEAP1 gene mutation, NFE2L2 /NRF2 mutation, genomic loss of KEAP1, reduced KEAP1 protein expression, increased NRF2 protein expression, and/or protein and/or gene expression modification in a gene or protein in the KEAP1/NRF2 pathway. In another aspect, the cancer cells further include a KRAS mutation and/or a STK11/LKB1 alteration.

In one embodiment, the invention provides a method of identifying a subject as a responder to a macrophage migration inhibitory factor (MIF) targeting agent therapy including identifying an alteration in a KEAP1/NRF2 pathway in a cancer cell in the subject, wherein the presence of the alteration is indicative of a subject who is a responder, and the absence of the alternation is indicative of a subject who is a non-responder, thereby identifying a subject as a responder to a MIF targeting agent therapy.

In one aspect, the alteration in the KEAP1/NRF2 pathway is selected from the group consisting of a KEAP1 gene mutation, NFE2L2/NRF2 mutation, genomic loss of KEAP1, reduced KEAP1 protein expression, increased NRF2 protein expression, and/or protein and/or gene expression modification in a gene or protein in the KEAP1/NRF2 pathway. In another aspect, the cancer cells further comprise a KRAS mutation and/or a STK11/LKB1 alteration. In one aspect, the method further includes administering to the subject a further therapeutic treatment. In some aspects, the treatment is selected from the group consisting of immune checkpoint inhibitor therapy, chemotherapy and radiotherapy.

### BRIEF DESCRIPTION OF THE DRAWINGS

FIGS. 1A-1D show that LKB1/STK11 and KEAP1 alterations are genomic determinants of immunotherapy resistance. FIG. 1A is a graph illustrating response rates to pemetrexed/platin/pembrolizumab (PCP) chemo-immunotherapy in STK11 and KEAP1-defined wild type (wt) and mutant subgroups. FIG. 1B is a graph illustrating response rates to pemetrexed/platin/pembrolizumab (PCP) chemo-immunotherapy in STK11 and KEAP1-defined wild type (wt) and mutant subgroups. FIG. 1C shows a Kaplan-Mayer graph illustrating progression free survival of STK11/LKB1 and KEAP1 mutant or wt defined patients. FIG. 1D a Kaplan-Mayer graph illustrating overall survival of STK11/LKB1 and KEAP1 mutant or wt defined patients.

FIGS. 2A-2C show that KEAP1 loss reduces response to immunotherapy in syngeneic Kras-mutant models (K). FIG. 2A is a graph illustrating tumor volumes of LKR13 K syngeneic mouse models dosed twice per week with 200 μg of anti-PD 1 antibody. FIG. 2B is a graph illustrating tumor volumes of LKR13 Kras-mutant and KEAP1 knock-out (KK) syngeneic mouse models dosed twice per week with 200 μg of anti-PD 1 antibody. FIG. 2C is a graph illustrating tumor volumes of LKR13 Kras-mutant plus LKB1 and KEAP1 knock-out (KLK) syngeneic mouse models dosed twice per week with 200 μg of anti-PD 1 antibody.

FIGS. 3A-3C show that KEAP1 loss induces TME modifications. scRNA-seq analysis of cell composition was performed across LKR13 K, KL, KK and KLK syngeneic mouse models. FIG. 3A is a graph illustrating the frequency of cell clusters. FIG. 3B is a graph illustrating two-dimensional projection (UMAP) of total cells based on scRNAseq analysis in LKR13 murine K, KL, KK and KLK tumors. FIG. 3C shows frequency of indicated immune cells.

FIGS. 4A-4C show that KEAP1-deficient tumors upregulate MIF expression and secretion. FIG. 4A is a graph illustrating the expression of MIF across KRAS subgroups in LUAD patients. FIG. 4B is a graph illustrating MIF expression in murine tumor cells from scRNAseq analysis in preclinical murine models. FIG. 4C is a graph illustrating MIF secretion in vitro for indicated murine cell lines.

FIGS. 5A-5C show the negative regulation of MIF Expression by KEAP1. FIG. 5A shows grows illustrating the analysis of MIF mRNA levels across LKR13 K, KK, KL, and KLK cell lines. FIG. 5B shows immunoblots illustrating the examination of MIF protein levels in LKR13 K, KK, KL, and KLK cell lines. FIG. 5C shows immunoblots illustrating the assessment of MIF protein levels following the overexpression of wild-type KEAP1 in human H2030, H23, and H2122 cell lines.

FIGS. 6A-6B show that MIF depletion reduces cell proliferation and increases PD-L1 expression in KEAP1-deficient tumor cells. FIG. 6A is a graph illustrating in vitro cell proliferation assayed by cell counting for K, KK, KL and KLK parental murine cells and their respective MIF-deficient clones. FIG. 6B is a graph illustrating PD-L1 protein levels in MIF KO clones and parental control K, KK, KL and KLK murine cells.

FIGS. 7A-7D show that MIF loss does not significantly reduce K and KL tumor growth but significantly reduces KK and KLK tumor growth in vivo. FIG. 7A is a graph illustrating tumor volume measured in syngeneic LKR13 K parental MIF-proficient and 3 MIF KO tumors in immunocompetent mice. FIG. 7B is a graph illustrating tumor volume measured in syngeneic LKR13 KL parental MIF-proficient and 3 MIF KO tumors in immunocompetent mice. FIG. 7C is a graph illustrating tumor volume measured in syngeneic LKR13 KK parental MIF-proficient and 3 MIF KO tumors in immunocompetent mice. FIG. 7D is a graph illustrating tumor volume measured in syngeneic LKR13 KLK parental MIF-proficient and 3 MIF KO tumors in immunocompetent mice.

FIGS. 8A-8B show that MIF loss enhances anti-tumor immune response. FIG. 8A is a graph illustrating tumor volume measured in KK MIF KO tumors in immunocompetent 129/Sv and athymic nude mice. FIG. 8B is a graph illustrating tumor volume measured in KLK MIF KO tumors in immunocompetent 129/Sv and athymic nude mice.

FIG. 9 shows that MIF loss improves response to IO in KLK tumors. FIG. 9 is a graph illustrating tumor volume measured in KLK parental or MIF KO syngeneic models in immunocompetent 129/Sv treated with vehicle or anti-PD1 immunotherapy at indicated dose schedule.

### DETAILED DESCRIPTION OF THE INVENTION

The present invention is based on the seminal discovery that an alteration in the KEAP1/NRF2 pathway in a cancer cell is associated with increased levels and/or secretion of macrophage migration inhibitory factor (MIF), which in turn can be targeted by MIF targeting agents to sensitize cancer cells to therapy, e.g., immune checkpoint inhibitors.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

As used herein, the term “about” in association with a numerical value is meant to include any additional numerical value reasonably close to the numerical value indicated. For example, and based on the context, the value can vary up or down by 5-10%. For example, for a value of about 100, means 90 to 110 (or any value between 90 and 110).

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

In one embodiment, the present invention provides a method of treating non-small cell lung cancer (NSCLC) in a subject including: a) identifying an alteration in a KEAP1/NRF2 pathway in a cancer cell in the subject, and b) administering to the subject identified with a KEAP1/NRF2 pathway alteration, an anti-macrophage migration inhibitory factor (MIF) agent, thereby treating NSCLC in the subject.

“Non-small-cell lung carcinoma” or “NSCLC” as used herein refers to any type of epithelial lung cancer other than small-cell lung carcinoma (SCLC). NSCLC accounts for about 80-85% of all lung cancers. As a class, NSCLCs are relatively insensitive to chemotherapy, compared to small-cell carcinoma. When possible, they are primarily treated by surgical resection with curative intent, although chemotherapy has been used increasingly both preoperatively (neoadjuvant chemotherapy) and postoperatively (adjuvant chemotherapy). The most common types of NSCLC are squamous-cell carcinoma, large-cell carcinoma, and adenocarcinoma. Adenocarcinoma of the lung is currently the most common type of lung cancer in “never smokers” (lifelong nonsmokers). Adenocarcinomas account for about 40% of lung cancers. Historically, adenocarcinoma was more often seen peripherally in the lungs than SCLC and squamous-cell lung cancer, both of which tend to be more often centrally located. Squamous-cell carcinoma (SCC) of the lung is more common in men than in women. It is closely correlated with a history of tobacco smoking, more so than most other types of lung cancer. Large-cell lung carcinoma (LCLC) is a heterogeneous group of undifferentiated malignant neoplasms originating from transformed epithelial cells in the lung. LCLCs typically comprise around 10% of all NSCLC, although newer diagnostic techniques seem to be reducing the incidence of diagnosis of “classic” LCLC in favor of more poorly differentiated SCCs and adenocarcinomas. LCLC is, in effect, a “diagnosis of exclusion”, in that the tumor cells lack light microscopic characteristics that would classify the neoplasm as a small-cell carcinoma, squamous-cell carcinoma, adenocarcinoma, or other more specific histologic type of lung cancer. LCLC is differentiated from SCLC primarily by the larger size of the anaplastic cells, a higher cytoplasmic-to-nuclear size ratio, and a lack of “salt-and-pepper” chromatin.

The term “subject” as used herein refers to any individual or patient to which the subject methods are performed. Generally, the subject is human, although as will be appreciated by those in the art, the subject may be a non-human animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” or “therapy” and refers to 1) therapeutic treatments or measures that cure, slow down, lessen symptoms of, and/or halt progression of a diagnosed pathologic conditions or disorder, and/or 2) prophylactic/preventative measures. Those in need of treatment may include individuals already having a particular medical disorder as well as those who may ultimately acquire the disorder (i.e., those needing preventive measures). The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., treatment of cancer). Such amount should be sufficient to inhibit tumor growth and increase subject's survival. The effective amount can be determined as described herein.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration.

The methods described herein include the identification of an alteration in a KEAP1/NRF2 pathway.

Oxidative stress plays an important role in the initiation and progression of many chronic diseases, including diabetes, cancer, and neurodegenerative diseases. Through the regulation of cytoprotective gene expression, the KEAP1-NRF2 stress response pathway is the principal inducible defense against oxidative and electrophilic stresses.

Nuclear factor (erythroid-derived 2)-like 2 (NRF2) is a basic leucine zipper (bZIP) transcription factor with a cap ‘n’ collar (CNC) structure. NRF2 transcription factor is ubiquitously expressed and present in various organs and tissues, including the kidney, muscle, lung, heart, liver and brain. It is tightly regulated by the repressor protein, KEAP1 (Kelch-like ECH-associated protein 1), in the cytoplasm, which subsequently plays an essential role in NRF2 degradation by the ubiquitin-proteasome pathway. Under oxidative stress, NRF2 dissociates from KEAP1, translocates to the nucleus and transactivates several cytoprotective genes to combat the oxidative stress.

KEAP1 is a dimeric protein consisting of 624 amino acid residues. KEAP1 acts as a substrate adapter protein for the E3 ubiquitin ligase complex formed by CUL3 and RBX1 and targets NRF2 for ubiquitination and degradation by the proteasome.

The KEAP1-NRF2 system is a vital member of regulating cells under a homeostatic environment. This system counters the xenobiotic and oxidative responses which defend the cells from external and internal toxicity. At basal homeostatic conditions, KEAP1 maintains a consistent generation of NRF2 and retains its low levels in the cytoplasm. A specific cysteine residue modification of KEAP1 is responsible for the conformational change of the protein in cells under oxidative stress conditions. This causes the initial detachment between the two proteins. Therefore, KEAP1-bound NRF2 is released and translocated inside the nucleus of the cell. The stress conditions lead to the suspension of KEAP1-NRF2 interactions and causes transcription of cytoprotective genes like NQO1, GSTs and GCL, which in turn, scavenges the cellular oxidative stress. According to another model, CUL3 gets dissociated from the KEAP1-CUL3 complex in the presence of ROS. Hence, ubiquitination of NRF2 is halted, which leads NRF2 to escape from the proteasomal degradation and results in its subsequent nuclear translocation.

The KEAP1-NRF2 complex triggers the degradation of NRF2 factor via CUL3-dependent E3-ubiquitin ligase-mediated ubiquitination and its successive degradation through proteasome. Cullin family proteins are hydrophobic in nature and have a high substrate specificity towards a multimeric complex of E3 ligases. Cullin proteins play an essential role by providing solid scaffolds to these E3 ligases, which polyubiquinates the substrate with the help of E2 ligases.

NRF2 activation upregulates the various set of enzymes for the detoxification of chemical carcinogens and confers protection against carcinogenicity, mutagenicity and other types of toxicity. NRF2 protects against oxidative stress, chemotherapeutic agents and radiotherapy. However, NRF2 disruption enables the cells towards carcinogens, which lead to the progression of inflammation and, finally, cancer formation. This dual action of NRF2 has been termed as a ‘double-edged sword’ with respect to the benefits or risks of the KEAP1-NRF2 pathway in cells. The excessive NRF2 expression leads to the survival of both normal and cancerous cells. Hence, the NRF2 downstream gene expression balance is required to obtain the clinical benefits and with less side effects. NRF2 has the ability to reduce the ROS and DNA damage in chemical-induced carcinogen cells.

Several genes in the pentose phosphate pathway including glucose-6-phosphate dehydrogenase (G6PD), phosphogluconate dehydrogenase (PGD), transketolase (TKT) and transaldolase 1(TALDO1 ), which are responsible for the regeneration of nicotinamide adenine dinucleotide phosphate (NADPH), as well as other metabolic genes, including malic enzyme 1(ME1 ), pyrophosphate phosphoribosyl amidotransferase (PPAT), methylenetetrahydrofolate dehydrogenase 2(MTHFD2 ) and isocitrate dehydrogenase 1(IDH1 ), were identified as transcription targets of NRF2. These proteins are responsible for purines synthesis, which are the building blocks of DNA and RNA, which in turn, leads to the proliferation of cancer cells. This supports the fact that elevated levels of NRF2 render cancer cells less sensitive to chemotherapeutic treatments and more resistant to a variety of anti-cancer agents.

As used herein the term “alteration in a KEAP1/NRF2 pathway” refers to any alteration or mechanism that leads to or is responsible for abnormally elevated levels of NRF2 in cancer cells.

Several mechanisms can lead to abnormally elevated levels of NRF2 in cancer cells, which includes:
(a) somatic mutation, including gain-of-function mutations in NFE2L2/NRF2, and loss-of-function mutations in KEAP1 and CUL3. Mutations in the KEAP1 gene have been identified in human lung adenocarcinoma cell lines, for example involving a glycine to cysteine substitution in the kelch domain of KEAP1. It exhibits reduced affinity of KEAP1 to the NRF2 and, thereby, activation of NRF2. Mutations in the ETGE and DLG motifs impair two-site substrate recognition of KEAP1, which leads to the stabilization of NRF2 and, subsequently, activation of target genes of NRF2. Somatic mutations in CUL3 were identified in hereditary type 2 papillary renal cell carcinoma.
(b) epigenetic silencing of KEAP1 by hypermethylation: epigenetic modification of KEAP1 promotes the activation of NRF2. Methylation in the promoter region of KEAP1 affects its expression and hinders the ability to bind to the NRF2. Therefore, this leads to the expression of NRF2.
(c) accumulation of p21 and p62 disrupts the NRF2-KEAP1 complex: it has been demonstrated that p53 negatively regulates NRF2 and specifically suppresses the transcription of target genes of NRF2. p21 (a direct downstream target of p53) associates with the DLG motif of NRF2, which leads to the disruption of KEAP1 binding with NRF2. As a result, NRF2 is stabilized in response to p21 upregulation. Another protein, p62 (sequestosome 1 protein), directly interacts with the kelch domain of KEAP1 via its STGE motif that is similar to the NRF2 ETGE motif, thereby disrupting the KEAP1-NRF2 complex. It causes a decrease in the ubiquitination of NRF2, an increase in NRF2 stability and, ultimately, leads to the enhanced expression of ARE-bearing genes.
(d) transcriptional upregulation of NRF2 by oncogenes: oncogenes like KRAS, BRAF and C-MYC increased the mRNA level of NFE2L2/NRF2 and target genes of NRF2. The C-MYC oncogene is involved in both increased and decreased expression of phase II antioxidant genes, depending on the pleiotropic effect. The oncogenic transmembrane protein MUC1-C transcribes the C-MYC mRNA and proteins, which in turn, upregulates the C-MYC gene expression. Upregulation of C-MYC genes leads to decrease of the NRF2 stability.
(e) metabolic activation of NRF2 by Krebs cycle intermediates: in Krebs cycle, fumarate modifies cysteine residues within KEAP1, which disrupt the ability to ubiquitinate NRF2. This leads to the prolonged activation of NRF2 (Adam et al. 2011).

In one aspect, identifying an alteration in a KEAP1/NRF2 pathway includes detecting a KEAP1 gene mutation, NFE2L2/NRF2 mutation, genomic loss of KEAP1, reduced KEAP1 protein expression, increased NRF2 protein expression, and/or protein and/or gene expression modification in a gene or protein in the KEAP1/NRF2 pathway in the cancer cell.

By “identifying an alteration in a KEAP1/NRF2 pathway” in a cancer cell, it is meant that a sample of the tumor is obtained from the patient and analyzed to detect any alteration in the KEAP1/NRF2 pathway.

A “sample” or “biological sample” is meant to refer to any “biological specimen” collected from a subject, and that is representative of the content or composition of the source of the sample (i.e., the tumor), considered in its entirety. A sample can be collected and processed directly for analysis or be stored under proper storage conditions to maintain sample quality until analyses are completed. Ideally, a stored sample remains equivalent to a freshly collected specimen. The source of the sample can be an internal organ, vein, artery, or even a fluid that contains cancer cells (e.g., circulating cancer cells). Non-limiting examples of sample include organ biopsy, tumor biopsy, cerebrospinal fluid (CSF), blood, plasma and urine.

The alteration can be detected by analyzing nucleic acids and/or in proteins in the sample. “Nucleic acid” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, or (iv) was synthesized, for example, by chemical synthesis. A nucleic can be employed for introduction into, i.e., transfection of, cells, in particular, in the form of RNA which can be prepared by in vitro transcription from a DNA template. The RNA can moreover be modified before application by stabilizing sequences, capping, and polyadenylation. “Peptide”, “polypeptide” and “protein” are used interchangeably herein and refer to any chain of at least two amino acids, linked by a covalent chemical bound. As used herein polypeptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

Non-limiting examples of detection methods include single-cell RNA sequencing, exome sequencing, targeted exome sequencing, immunohistochemistry, immunofluorescence, and any methods known in the art suitable for the detection and analysis of nucleic acid and protein alteration in a cell.

In some aspects, in addition to an alteration in the KEAP1/NRF2 pathway, the cancer cell includes one or more additional mutations and/or alteration in others genes/pathways, including KRAS gene, and the STK11/LKB1 pathway. In another aspect, the cancer cell further includes a KRAS mutation and/or a STK11/LKB1 alteration. For the example, the cancer cell includes (i) an alteration in the KEAP1/NRF2 pathway and a KRAS mutation, (ii) an alteration in the KEAP1/NRF2 pathway and an alteration in the STK11/LKB1 pathway, or (iii) an alteration in the KEAP1/NRF2 pathway, a KRAS mutation and an alteration in STK11/LKB1.

KRAS (Kirsten rat sarcoma virus) is a gene that encode a K-Ras protein, which is a part of the RAS/MAPK pathway. The protein relays signals from outside the cell to the cell's nucleus to instruct the cell to proliferate or to mature and differentiate. The K-Ras protein is a GTPase, a class of enzymes which convert the nucleotide guanosine triphosphate (GTP) into guanosine diphosphate (GDP). In this way the K-Ras protein acts like a switch that is turned on and off by the GTP and GDP molecules. To transmit signals, it must be turned on by binding to a molecule of GTP. The K-Ras protein is turned off when it converts the GTP to GDP. When the protein is bound to GDP, it does not relay signals to the cell's nucleus. Once it is allosterically activated, KRAS recruits and activates proteins necessary for the propagation of growth factors, as well as other cell signaling receptors like c-Raf and PI 3-kinase. KRAS upregulates the GLUT1 glucose transporter, thereby contributing to the Warburg effect in cancer cells. KRAS binds to GTP in its active state. It also possesses an intrinsic enzymatic activity which cleaves the terminal phosphate of the nucleotide, converting it to GDP. Upon conversion of GTP to GDP, KRAS is deactivated. The rate of conversion is usually slow but can be increased dramatically by an accessory protein of the GTPase-activating protein (GAP) class.

KRAS is a proto-oncogene; a single amino acid substitution, and in particular a single nucleotide substitution, is responsible for an activating mutation. The transforming protein that results is implicated in various malignancies, including lung adenocarcinoma, mucinous adenoma, ductal carcinoma of the pancreas and colorectal cancer. Somatic KRAS mutations are found at high rates in leukemias, colorectal cancer, pancreatic cancer and lung cancer. Whether a patient is positive or negative for a mutation in the epidermal growth factor receptor (EGFR) will predict how patients will respond to certain EGFR antagonists such as erlotinib (Tarceva ®) or gefitinib (Iressa®). Patients who harbor an EGFR mutation have a 60% response rate to erlotinib. However, the mutation of KRAS and EGFR are generally mutually exclusive. Lung cancer patients who are positive for KRAS mutation (and the EGFR status would be wild type) have a low response rate to erlotinib or gefitinib estimated at 5% or less.

Serine/threonine kinase 11(STK11 ) gene codifies for liver kinase B1 (LKB1) protein or renal carcinoma antigen NY-REN-19 is a protein kinase that in humans is encoded by the STK11 gene. The STK11/LKB1 gene, which encodes a member of the serine/threonine kinase family, regulates cell polarity and functions as a tumor suppressor. LKB1 is a primary upstream kinase of adenosine monophosphate-activated protein kinase (AMPK), a necessary element in cell metabolism that is required for maintaining energy homeostasis. LKB1 exerts its growth suppressing effects by activating a group of ˜14 other kinases, comprising AMPK and AMPK-related kinases. Activation of AMPK by LKB1 suppresses growth and proliferation when energy and nutrient levels are scarce. Activation of AMPK-related kinases by LKB1 plays vital roles maintaining cell polarity thereby inhibiting inappropriate expansion of tumor cells. Loss of LKB1 leads to disorganization of cell polarity and facilitates tumor growth under energetically unfavorable conditions. For example, loss of LKB1 activity is associated with highly aggressive HER2+ breast cancer. At least 51 disease-causing mutations in this gene have been discovered.

After the identification of an alteration in a KEAP1/NRF2 pathway, and optionally of a further mutation in KRAS and or an alteration in the STK11/LKB1 gene, the method includes administering to the subject identified with a KEAP1/NRF2 pathway alteration, an anti-macrophage migration inhibitory factor (MIF) agent.

Macrophage migration inhibitory factor (MIF) is a proinflammatory cytokine that has been implicated in the pathogenesis of chronic inflammatory diseases. MIF is expressed by a number of cell types such as macrophage cells, lymphocytes, neutrophils and eosinophils. In addition, the important role for this proinflammatory cytokine is now known in cancer. MIF is expressed by a variety of cancers including prostate, colon, liver and lung, and a number of protumor functions have been assigned to this protein. These functions include the downregulation of the tumor suppressor p53 and prevention of p53-induced apoptosis by MIF, thereby facilitating malignant transformation. Specific studies in lung cancer have identified MIF as a key regulator of tumor growth. MIF expression has been shown to correlate with expression of angiogenic chemokines in non-small cell lung cancer. MIF also promotes constitutive extracellular signal-regulated kinase (ERK) activation mirroring the actions of oncogenes such as RAS. MIF has been found to support hypoxic adaptation of cells by inducing stabilization of hypoxia-inducible factor 1-α (HIF1α), and readily contributes to a microenvironment favoring tumor growth and proliferation by promoting angiogenesis required to sustain tumor growth.

The tumor microenvironment (TME) is an essential component of the tumor structure. The crosstalk between stromal cells and those of the immune system provides an important balance in the control of neoplasia development. Immunological escape is a key step in establishment and maintenance of tumor progression. The interaction between cells in the microenvironment is mainly mediated by secretion of pro-tolerogenic factors resulting in functional losses of cells of innate and adaptive immunity. MIF has been considered an important pro-tolerogenic factor secreted in the TME. Following its secretion, in the TME, MIF can bind to its receptor CD74, which is expressed on tumor associated macrophages, dendritic cells (DC), regulatory T cells (Tregs) and myeloid-derived suppressor cells (MDSC), promoting immunological escape and tumor growth. In addition to its immunosuppressive effects on innate immune cells, MIF can also modulate the adaptive immune response through suppression of cytotoxic T lymphocytes (CTL).

When extracellular MIF binds to its primary receptor cluster of differentiation 74 (CD74) on the cell membrane, co-receptors including CD44 or C-X-C chemokine receptors (CXCRs; CXCR2, CXCR4, and CXCR7) are also required to activate downstream signaling pathways. Once the CD74/CD44 complex is activated by MIF through the proto-oncogene tyrosine-protein kinase (SRC), mitogen-activated protein kinase (MAPK) family members such as the extracellular signal-related kinase ½(ERK½), phosphoinositide 3-kinase (PI3K), and protein kinase B (PKB, also known as AKT) are phosphorylated and subsequently activated. Sustained ERK½ activation promotes cancer cell invasion and inhibits cell death. AKT activation leads to phosphorylation of the proapoptotic proteins including Bel-2 agonist of cell death (BAD) and transcription factor forkhead box O-3a (FoxO3a), promoting cancer cell survival. MIF-induced cyclo-oxygenase-2 (COX-2)/prostaglandin E2 (PGE-2) activation enhances tumor growth, cancer cell viability, and metastasis. MIF downregulates tumor suppressor protein p53, leading to inhibition of p53-dependent apoptosis, accumulation of mutation, and proliferation of cancer cells. ERK½ and PI3K/AKT also activate transcription factors including nuclear factor-kappa B (NF-κB) and activator protein-1 (AP1), which result in the release of pro-inflammatory cytokines such as IL-6, IL-8, IL-10, and TNF-α. IL-6 and IL-8 also exhibit pro-tumorigenic functions including promotion of tumor formation by enhancing proliferation, reducing apoptosis, and promoting invasiveness. Besides, MIF inhibits p53-mediated apoptosis in macrophage with the induction of increased cytoplasmic phospholipase A2 (PLA2), arachidonic acid, COX2 and PGE2, which sustains the macrophage pro-inflammatory function (Mitchell. et al., 1999; Mitchell et al., 2002).

As used herein, the term anti-MIF agent is meant to refer to any agent that inhibits one or more of the intra-and/or extra-cellular activities of MIF, including its binding to its receptors, its secretion, its enzymatic activity, etc.

In one aspect, the MIF targeting agent is selected from the group consisting of an anti-MIF antibody, anti-MIF isoform antibody, anti-oxidized MIF antibody, anti-MIF receptor antibody, MIF receptor inhibitor, CD74 inhibitor, MIF blocking peptide, CAR-T cell, anti-MIF antibody drug conjugate and anti-MIF small molecule inhibitor.

In one aspect, the anti-MIF agent is a binding peptide such as an anti-MIF antibody.

The term “binding peptide”, “binding protein”, and the like, as used herein are meant to refer to any peptide or polypeptide (including proteins and fusion proteins) that can bind to a protein of interest. Binding selectivity or selective binding can be defined with respect to the binding of a binding protein or antibody to a substrate (e.g., a protein like MIF) forming a complex. Binding selectivity describes how a binding protein can bind more preferentially to one protein (e.g., MIF) than another protein. The binding proteins described herein can include at least one CDR region. By “CDR region” it is meant that the binding protein include one or more of the three segments called CDRs or hypervariable regions and a more highly conserved portions of the variable domains of an antibody that specifically recognize the protein of interest. The binding proteins can be an antibody, an antibody fragment, and the like, having specific binding to one or more target polypeptide, including the protein of interest (MIF).

The term “antibody” or “binding protein” generally refers to immunoglobulin molecules and immunologically active portions of immunoglobulin molecules, i.e., molecules that contain an antigen binding site that immunospecifically binds an antigen. “Native antibodies” and “intact immunoglobulins”, or the like, are usually heterotetrameric glycoproteins of about 150,000 daltons, composed of two identical light (L) chains and two identical heavy (H) chains. The light chains from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called α, δ, ε, γ, and μ, respectively.

Antibodies can be cleaved experimentally with the proteolytic enzyme papain, which causes each of the heavy chains to break, producing three separate antibody fragments. The two units that consist of a light chain and a fragment of the heavy chain approximately equal in mass to the light chain are called the Fab fragments (i.e., the “antigen binding” fragments). The third unit, consisting of two equal segments of the heavy chain, is called the Fc fragment. The Fc fragment is typically not involved in antigen-antibody binding but is important in later processes involved in ridding the body of the antigen. As used herein, “antibody fragments” include a portion of an intact antibody including the antigen binding or variable region of the intact antibody. Examples of antibody fragments include Fab, Fab′ and F(ab′)2, Fc fragments or Fc-fusion products, single-chain Fvs (scFv), disulfide-linked Fvs (sdfv) and fragments including either a VL or VH domain; diabodies, tribodies and the like (Zapata et al. Protein Eng. 8(10):1057-1062 [1995]).

Non-limiting examples of anti-MIF antibodies include: (i) nanobodies such as NbE5 and NbE10; (ii) monoclonal antibodies such as those developed at Baxter including BaxG03, BaxB01, and BaxM159. Bax69 (imalumab) is currently investigated in solid tumors and metastatic colorectal adenocarcinoma; (iii) antibodies targeting different isoforms of MIF, such as oxidized MIF (oxMIF), including the antibodies developed by OncoOne (ON104, ON203,ON102, and ON-05). oxMIF is a disease-related isoform of MIF, which is generated by a post-translational modification of MIF in inflammatory processes and tumorigenesis. Unlike MIF, oxMIF is differentially expressed in healthy and diseased tissue and can be detected in inflamed tissue and solid tumors. Importantly, the post-translational modification leads to a structural transformation that exposes epitopes in the MIF homotrimer that are otherwise inaccessible to antibodies in the center of the trimer; and (iv) antibodies or binding peptides targeting MIF receptors. As MIF relies largely on CD74 to regulate the downstream cellular events, treatment targeting CD74 holds great potential to inhibit MIF signaling. Humanized anti-CD74 monoclonal antibody, such as milatuzumab are therefore interesting antibodies candidates to target MIF.

In one aspect, the anti-MIF agent is an anti-MIF antibody drug conjugate. The term “antibody drug conjugate” can be used interchangeably with the term “immunoconjugate” or “conjugate” as used herein and refers to a compound or a derivative thereof, such as a drug (i.e., an anti-cancer drug) that is linked to a cell binding agent (i.e., an anti-MIF antibody or fragment thereof) and is defined by a generic formula: C-L-A, wherein C=cytotoxin, L=linker, A=cell binding agent or antibody (i.e., anti-MIF antibody or antibody fragment thereof). Immunoconjugates can also be defined by the generic formula in reverse order: A-L-C.

In one aspect, the anti-MIF agent is a small molecule inhibitor. Small-molecule inhibitors of MIF primarily focus on rational structure-based design that target the MIF tautomerase activity and MIF-CD74 binding. Such small molecule MIF inhibitors may for example be identified using high-throughput clinically relevant MIF bioassay that can be applied for second-pass screening, glucocorticoid override, cellular proliferation, and cytokine release detection to expedite the discovery of efficient small molecular MIF inhibitors. Non-limiting examples of such small molecule inhibitor include ISO-1, SCD-19 and 4-IPP. Other anti-MIF small molecules include naphthalene derivatives which inhibit the cytokine or biological activity of microphage MIF, such as those describes in US Patent Application Publication No.: 2006/0106102A1, the disclosure of which is incorporated herewith in its entirety.

In one aspect, the anti-MIF agent is a MIF blocking peptide.

“Protein mimotopes”, or “blocking peptides” as used herein are small therapeutic peptides that prevent protein-protein interactions by selectively mimicking a native binding domain. A blocking peptide competitively inhibits protein-protein interaction by mimicking one of their binding domains, or “binding epitopes.” Initially, the term mimicking epitope, or “mimotope,” is meant to refer to a peptide macromolecule that reproduced, or “mimicked,” the epitope structure that elicits the same specific antibody response as the intact macromolecule from which it was derived. The term mimotope was expanded to include peptides that competitively interrupt protein-protein interactions by binding to one of the partner's binding domains, including those that interfere with protein-protein interactions, of both extra-and intracellular proteins. Blocking peptides have also been referred to as “peptide aptamers”. The term “aptamers” includes small nucleic acid and peptide ligands designed to interfere with RNA function. By design, small peptide aptamers, or peptide mimotopes, possess many of the attractive features of monoclonal antibodies, including high specificity and ligand binding affinity [with IC50 as low as the picomolar range]. Peptides are much smaller and require less time to synthesize than monoclonal antibodies and can be targeted to the extra-and intracellular compartments.

In one aspect, the anti-MIF agent is a CAR-T cell. Adoptive transfer of genetically engineered T cells, modified to recognize and eliminate cancer cells specifically can be engineered to target cancer cell that specifically express MIF. For example, T cells can be genetically modified to stably express on their surface chimeric antigen receptors (CAR). CAR are synthetic proteins comprising of a signaling endodomain, consisting of an intracellular domain of the CD3-zeta chain, a transmembrane domain, and an extracellular domain consisting of the antigen recognition fragment of a monoclonal antibody which gives the receptor its specificity for a tumor associated antigen (e.g., an scFv, or single chain variable region fragment). Upon interaction with the target cancer cell expressing the scFv' s cognate antigen, the chimeric antigen receptor triggers an intracellular signaling leading to T-cell activation and to a cytotoxic immune response against tumor cells. Such therapies have been shown to be efficient against relapsed/refractory disease. Additionally, CAR-T cells can be engineered to include co-stimulatory receptor that enhance the T-cell-mediated cytotoxic activity. Furthermore, CAR-T cells can be engineered to produce and deliver protein or agent of interest in the tumor microenvironment. An example of such a CAR-T cell includes a CAR-T cell presenting an engineered receptor that include a MIF-antigen recognition, such that cancer cell expressing MIF can be specifically targeted.

In another aspect, the method further includes administering to the subject a further therapeutic treatment.

The administration of the MIF targeting agent can be in combination with one or more additional therapeutic agents. The phrases “combination therapy”, “combined with” and the like refer to the use of more than one medication or treatment simultaneously to increase the response. The MIF targeting agent might for example be used in combination with other drugs or therapeutic treatment in use to treat cancer, and specifically NSCLC. Specifically, the administration of the MIF targeting agent to a subject can be in combination with lung-cancer therapy. Such therapies can be administered prior to, simultaneously with, or following administration of the MIF targeting agent.

The term “therapeutic treatment” is meant to include “anti-cancer therapy” or “anti-cancer treatment” which refer to any treatment that can be used to treat cancer, such as surgery, radiotherapy, chemotherapy, immunotherapy, and checkpoint inhibitor therapy. In some aspects, the therapeutic treatment is selected from the group consisting of immune checkpoint inhibitor therapy, chemotherapy and radiotherapy.

Examples of chemotherapy include treatment with a chemotherapeutic, cytotoxic or antineoplastic agents including, but not limited to, (i) anti-microtubules agents comprising vinca alkaloids (vinblastine, vincristine, vinflunine, vindesine, and vinorelbine), taxane (cabazitaxel, docetaxel, larotaxel, ortataxel, paclitaxel, and tesetaxel), epothilones (ixabepilone), and podophyllotoxin (etoposide and teniposide); (ii) antimetabolite agents comprising anti-folates (aminopterin, methotrexate, pemetrexed, pralatrexate, and raltitrexed), and deoxynucleoside analogues (azacitidine, capecitabine, carmofur, cladribine, clofarabine, cytarabine, decitabine, doxifluridine, floxuridine, fludarabine, fluorouracil, gemcitabine, hydroxycarbamide, mercaptopurine, nelarabine, pentostatin, tegafur, and thioguanine); (iii) topoisomerase inhibitors comprising Topoisomerase I inhibitors (belotecan, camptothecin, cositecan, gimatecan, exatecan, irinotecan, lurtotecan, silatecan, topotecan, and rubitecan) and Topoisomerase II inhibitors (aclarubicin, amrubicin, daunorubicin, doxorubicin, epirubicin, etoposide, idarubicinm, merbarone, mitoxantrone, novobiocin, pirarubicin, teniposide, valrubicin, and zorubicin); (iv) alkylating agents comprising nitrogen mustards (bendamustine, busulfan, chlorambucil, cyclophosphamide, estramustine phosphate, ifosamide, mechlorethamine, melphalan, prednimustine, trofosfamide, and uramustine), nitrosoureas (carmustine (BCNU), fotemustine, lomustine (CCNU), N-Nitroso-N-methylurea (MNU), nimustine, ranimustine semustine (MeCCNU), and streptozotocin), platinum-based (cisplatin, carboplatin, dicycloplatin, nedaplatin, oxaliplatin and satraplatin), aziridines (carboquone, thiotepa, mytomycin, diaziquone (AZQ), triaziquone and triethylenemelamine), alkyl sulfonates (busulfan, mannosulfan, and treosulfan), non-classical alkylating agents (hydrazines, procarbazine, triazenes, hexamethylmelamine, altretamine, mitobronitol, and pipobroman), tetrazines (dacarbazine, mitozolomide and temozolomide); (v) anthracyclines agents comprising doxorubicin and daunorubicin. Derivatives of these compounds include epirubicin and idarubicin; pirarubicin, aclarubicin, and mitoxantrone, bleomycins, mitomycin C, mitoxantrone, and actinomycin; (vi) enzyme inhibitors agents comprising FI inhibitor (Tipifarnib), CDK inhibitors (Abemaciclib, Alvocidib, Palbociclib, Ribociclib, and Seliciclib), PrI inhibitor (Bortezomib, Carfilzomib, and Ixazomib), PhI inhibitor (Anagrelide), IMPDI inhibitor (Tiazofurin), LI inhibitor (Masoprocol), PARP inhibitor (Niraparib, Olaparib, Rucaparib), HDAC inhibitor (Belinostat, Panobinostat, Romidepsin, Vorinostat), and PIKI inhibitor (Idelalisib); (vii) receptor antagonist agent comprising ERA receptor antagonist (Atrasentan), Retinoid X receptor antagonist (Bexarotene), Sex steroid receptor antagonist (Testolactone); (viii) ungrouped agent comprising Amsacrine, Trabectedin, Retinoids (Alitretinoin Tretinoin) Arsenic trioxide, Asparagine depleters (Asparaginase/Pegaspargase), Celecoxib, Demecolcine Elesclomol, Elsamitrucin, Etoglucid, Lonidamine, Lucanthone, Mitoguazone, Mitotane, Oblimersen, Omacetaxine mepesuccinate, and Eribulin.

Examples of immunotherapy include treatment with antibodies including, but not limited to, alemtuzumab, Avastin ® (bevacizumab), Bexxar® (tositumomab), CDP 870, and CEA-Scan (arcitumomab), denosumab, Erbitux® (cetuximab), Herceptin® (trastuzumab), Humira® (adalimumab), IMC-IIF 8, LeukoScan® (sulesomab), MabCampath® (alemtuzumab), MabThera® (rituximab), matuzumab, Mylotarg® (gemtuzumab oxogamicin), natalizumab, NeutroSpec® (Technetium (99mTc) fanolesomab), panitumamab, Panorex ® (edrecolomab), ProstaScint® (Indium-Ill labeled Capromab Pendetide), Raptiva® (efalizumab), Remicade ® (infliximab), ReoPro® (abciximab), rituximab, Simulect® (basiliximab), Synagis® (palivizumab), TheraCIM hR3®, tocilizumab, Tysabri® (natalizumab), Verluma® (nofetumomab), Xolair® (omalizumab), Zenapax® (dacliximab), Zevalin® (ibritumomab tiuxetan (IDEC-Y2B8) conjugated to yttrium 90), Gilotrif® (afatinib), Lynparza ® (olaparib), Perjeta ® (pertuzumab), Otdivo® (nivolumab), Bosulif® (bosutinib), Cabometyx® (cabozantinib), trastuzumab-dkst (Ogivri®), Sutent® (sunitinib malate), Adcetris® (brentuximab vedotin), Alecensa® (alectinib), Calquence® (acalabrutinib), Yescarta® (ciloleucel), Verzenio ® (abemaciclib), Keytruda® (pembrolizumab), Aliqopa® (copanlisib), Nerlynx® (neratinib), Imfinzi® (durvalumab), Darzalex® (daratumumab), Tecentriq® (atezolizumab), Tarceva ® (erlotinib), and Mobocertinib (Exkivity®).

More than one kind of treatment is often used, depending on the stage of the lung cancer, the individual's overall health, age, response to chemotherapy, and other factors such as the likely side effects of the treatment. After full staging, NSCLC patient can typically be classified in one of three different categories: patients with early, nonmetastatic disease (stages I and II, and select type III tumors), patients with locally advanced disease confined to the thoracic cavity (e.g., large tumors, tumors involving critical chest structures, or patients with positive mediastinal lymph nodes), or patients with distant metastasis outside of the thoracic cavity. Lung cancer-specific treatment may be defined based on such combination of factors.

NSCLCs are usually not very sensitive to chemotherapy and/or radiation, so surgery (lung resection to remove the tumor) remains the treatment of choice if patients are diagnosed at an early stage. If the persons have a small, but inoperable tumor, they may undergo highly targeted, high-intensity radiation therapy. New methods of giving radiation treatment allow doctors to be more accurate in treating lung cancers. This means less radiation affects nearby healthy tissues. New methods include Cyberknife and stereotactic body radiation therapy. Certain people who are deemed to be higher risk may also receive adjuvant (ancillary) chemotherapy after initial surgery or radiation therapy. A number of possible chemotherapy agents can be selected, but most involve the platinum-based chemotherapy e.g., cisplatin and carboplatin.

Two genetic markers are routinely profiled in NSCLC tumors to guide further treatment decision-making-mutations within epidermal growth factor (EGFR) and anaplastic lymphoma kinase. Also, a number of additional genetic markers are known to be mutated within NSCLC and may impact treatment in the future, including BRAF, HER2/neu, and KRAS. For advanced NSCLC, a combined chemotherapy treatment approach that includes cetuximab, an antibody that targets the EGFR signaling pathway, is more effective at improving a person's overall survival when compared to standard chemotherapy alone. Thermal ablations, i.e., RFA, cryoablation, and microwave ablation, are appropriate for palliative treatment of tumor-related symptoms or recurrences within treatment fields. NSCLC patients with EML4-ALK translocations or mutations in the ROSI gene; may benefit from ALK inhibitors, which are now approved for this subset of patients. Crizotinib, which gained FDA approval in August 2011, is an inhibitor of several kinases, specifically ALK, ROS1, and MET. Crizotinib has been shown in clinical studies to have response rates around 60% if patients are shown to have ALK-positive disease.

NSCLC patients with advanced disease who are not found to have either EGFR or ALK mutations may receive bevacizumab, which is a monoclonal antibody medication targeted against the vascular endothelial growth factor (VEGF).

Non-small cell lung cancer (NSCLC) cells expressing programmed death-ligand 1 (PD-L1) could interact with programmed death receptor 1 (PD-1) expressed on the surface of T cells, and result in decreased tumor cell kill by the immune system. Anti PD-L1 monoclonal antibodies and anti PD-1 monoclonal antibodies could interact with programmed death receptor 1 (PD-1) expressed on the surface of T cells, and result in decreased tumor cell kill by the immune system. The FDA approved pembrolizumab for the treatment of metastatic NSCLC in patients whose tumors express PD-L1 and who have failed treatment with other chemotherapeutic agents.

“Checkpoint inhibitor therapy” is a form of cancer treatment currently that uses immune checkpoints which affect immune system functioning. Immune checkpoints can be stimulatory or inhibitory. Tumors can use these checkpoints to protect themselves from immune system attacks. Checkpoint therapy can block inhibitory checkpoints, restoring immune system function. Checkpoint proteins include programmed cell death 1 protein (PDCD1, PD-1; also known as CD279) and its ligand, PD-1 ligand 1 (PD-L1, CD274), cytotoxic T-lymphocyte-associated protein 4 (CTLA-4), A2AR (Adenosine A2A receptor), B7-H3 (or CD276), B7-H4 (or VTCN1), BTLA (B and T Lymphocyte Attenuator, or CD272), IDO (Indoleamine 2,3-dioxygenase), KIR (Killer-cell Immunoglobulin-like Receptor), LAG3 (Lymphocyte Activation Gene-3), TIM-3 (T-cell Immunoglobulin domain and Mucin domain 3), and VISTA (V-domain Ig suppressor of T cell activation).

Programmed cell death protein 1, also known as PD-1 and CD279 (cluster of differentiation 279), is a cell surface receptor that plays an important role in down-regulating the immune system and promoting self-tolerance by suppressing T cell inflammatory activity. PD-1 is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells).

PD-1 has two ligands, PD-L1 and PD-L2, which are members of the B7 family. PD-L1 protein is upregulated on macrophages and dendritic cells (DC) in response to LPS and GM-CSF treatment, and on T cells and B cells upon TCR and B cell receptor signaling, whereas in resting mice, PD-L1 mRNA can be detected in the heart, lung, thymus, spleen, and kidney. PD-L1 is expressed on almost all murine tumor cell lines, including PA1 myeloma, P815 mastocytoma, and B16 melanoma upon treatment with IFN-γ. PD-L2 expression is more restricted and is expressed mainly by DCs and a few tumor lines.

CTLA4 or CTLA-4 (cytotoxic T-lymphocyte-associated protein 4), also known as CD152 (cluster of differentiation 152), is a protein receptor that, functioning as an immune checkpoint, downregulates immune responses. CTLA4 is constitutively expressed in regulatory T cells but only upregulated in conventional T cells after activation—a phenomenon which is particularly notable in cancers. CTLA4 is a member of the immunoglobulin superfamily that is expressed by activated T cells and transmits an inhibitory signal to T cells. CTLA4 is homologous to the T-cell co-stimulatory protein, CD28, and both molecules bind to CD80 and CD86, also called B7-1 and B7-2 respectively, on antigen-presenting cells. CTLA-4 binds CD80 and CD86 with greater affinity and avidity than CD28 thus enabling it to outcompete CD28 for its ligands. CTLA4 transmits an inhibitory signal to T cells, whereas CD28 transmits a stimulatory signal. CTLA4 is also found in regulatory T cells and contributes to its inhibitory function. T cell activation through the T cell receptor and CD28 leads to increased expression of CTLA-4.

There are several checkpoint inhibitors that are currently used to treat cancer. PD-1 inhibitors include Pembrolizumab (Keytruda®) and Nivolumab (Opdivo®). PD-L1 inhibitors include Atezolizumab (Tecentriq®), Avelumab (Bavencio®) and Durvalumab (Imfinzi®). CTLA-4 inhibitors include Iplimumab (Yervoy®). There are several other checkpoint inhibitors being developed including an anti B7-H3 antibody (MGA271), an anti-KIR antibody (Lirilumab) and an anti-LAG3 antibody (BMS-986016).

In various aspects, the immune checkpoint inhibitor therapy includes a PD-1 inhibitor and/or a PD-L1 inhibitor.

In many aspects, the PD-1 inhibitor and/or a PD-L1 inhibitor are selected from the group consisting of nivolumab, pembrolizumab, atezolizumab, avelumab, durvalumab, cemiplimab, and dostarlimab.

In one aspect, treating NSCLC includes inhibiting NSCLC cell proliferation, reducing NSCLC tumor growth and/or increasing NSCLC patient survival.

The present invention relies in part on the discovery that MIF loss is associated with a robust reduced cell proliferation rate in tumor cells lacking KEAP1 expression. Therefore, by targeting MIF expression and/or MIF secretion using a MIF targeting agent, the methods described herein reduce, slow down and/or completely inhibit the proliferation of cancer cells having an alteration in the KEAP1/NRF2 pathway, wherein such alteration is responsible for the reduce expression of KEAP1 and/or increased expression of NRF2.

In another embodiment, the invention provides a method of reducing immune checkpoint therapy resistance, increasing immune checkpoint therapy sensitivity and/or increasing anti-tumor activity in a subject having cancer including administering to the subject an anti-macrophage migration inhibitory factor (MIF) agent, wherein the subject's cancer cells have a KEAP1/NRF2 alteration, thereby reducing immune checkpoint therapy resistance, increasing immune checkpoint therapy sensitivity and/or increasing anti-tumor activity in the subject.

“Immune checkpoint therapy resistance” refers to both primary and acquired resistance to checkpoint inhibitor therapies. Resistance occurs when cancer cells do not respond to immunotherapy. The resistance can be primary, when the cells never response to the therapy, or acquired, which occurs as the disease progresses after an initial response (which may be broadly considered as including stabilization of the disease, as well as objective responses, regardless of treatment duration time and treatment discontinuation). As used herein, the term “reducing immune checkpoint therapy resistance” is meant refer to any inhibition of the immune checkpoint therapy resistance, whether primary or acquired, and includes stabilizing a disease that was otherwise progressing under checkpoint inhibitor therapy and instating or re-instating sensitivity of the cancer cell to an immune checkpoint inhibitor.

When cancer cells are resistant to checkpoint inhibitor therapy, they are not sensitive to such treatment. The methods described herein instate or re-instate sensitivity of the cancer cell to immune checkpoint inhibitor. As such, the term “increasing” sensitivity is meant to refers to both rendering cancer cells that were never sensitive to a checkpoint inhibitor therapy, sensitive to it, and to re-sensitizing cancer cells that were sensitive but that lost that sensitivity along the course of the treatment and/or natural evolution of the disease.

In another aspect, reducing immune checkpoint therapy resistance and/or increasing immune checkpoint therapy sensitivity includes increasing cancer cell PD-L1 expression, decreasing MIF level and/or MIF secretion, and/or increasing anti-tumor activity.

The present invention relies in part on the discovery that depletion of MIF induces PD-L1 expression in cancer cells. Therefore, by targeting MIF expression and/or MIF secretion using a MIF targeting agent, the methods described herein which decrease MIF level and/or MIF secretion induce or increase the expression of PD-L1 by cancer cells.

In one aspect, the immune checkpoint therapy includes a PD-1 inhibitor and/or a PD-L1 inhibitor. In some aspects, the PD-1 inhibitor and/or a PD-L1 inhibitor are selected from the group consisting of nivolumab, pembrolizumab, atezolizumab, avelumab, durvalumab, cemiplimab, and dostarlimab.

In another aspect, increasing anti-tumor activity includes reversing MIF-induced cancer cell immunosuppressed phenotype, increasing cancer cell sensitivity to immune checkpoint blockade, inhibiting tumor-associated immune evasion, increasing tumor immune infiltration and/or inhibiting pro-tumorigenic M2 macrophage polarization.

The term “immune response” refers to an integrated bodily response to an antigen and preferably refers to a cellular immune response or a cellular as well as a humoral immune response. The immune response may be protective/preventive/prophylactic and/or therapeutic. The immune system is a system of biological structures and processes within an organism that protects against disease. This system is a diffuse, complex network of interacting cells, cell products, and cell-forming tissues that protects the body from pathogens and other foreign substances, destroys infected and malignant cells, and removes cellular debris: the system includes the thymus, spleen, lymph nodes and lymph tissue, stem cells, white blood cells, antibodies, and lymphokines. B cells or B lymphocytes are a type of lymphocyte in the humoral immunity of the adaptive immune system and are important for immune surveillance. T cells or T lymphocytes are a type of lymphocyte that plays a central role in cell-mediated immunity. There are two major subtypes of T cells: the killer T cell and the helper T cell. In addition, there are suppressor T cells which have a role in modulating immune response. Killer T cells only recognize antigens coupled to Class I MHC molecules, while helper T cells only recognize antigens coupled to Class II MHC molecules. These two mechanisms of antigen presentation reflect the different roles of the two types of T cell. A third minor subtype are the γδ T cells that recognize intact antigens that are not bound to MHC receptors. In contrast, the B cell antigen-specific receptor is an antibody molecule on the B cell surface and recognizes whole pathogens without any need for antigen processing. Each lineage of B cell expresses a different antibody, so the complete set of B cell antigen receptors represent all the antibodies that the body can manufacture.

The terms “immunoreactive cell” “immune cells” or “immune effector cells” in the context of the present invention relate to a cell which exerts effector functions during an immune reaction, such as an anti-cancer immune response (i.e., anti-cancer immune activity). An “immunoreactive cell” preferably is capable of binding an antigen or a cell characterized by presentation of an antigen, or an antigen peptide derived from an antigen and mediating an immune response. For example, such cells secrete cytokines and/or chemokines, secrete antibodies, recognize cancerous cells, and optionally eliminate such cells. For example, immunoreactive cells comprise T cells (cytotoxic T cells, helper T cells, tumor infiltrating T cells), B cells, natural killer cells, neutrophils, macrophages, and dendritic cells.

“Anti-cancer immune activity” as used herein refers to any aspects of an immune response that can be induced against a cancer cell. Cancer cells are well known for developing evasion mechanisms to escape anti-cancer immune activity. By “increasing anti-tumor activity” it is meant that the methods described herein suppress, reverse, or inhibit immune evasion mechanism developed by cancer cells. Non-limiting examples of evasion mechanisms include induction of an immunosuppressed phenotype, inhibition of tumor immune infiltration, and polarization of pro-tumorigenic M2 macrophages. Such evasion mechanisms are induced at least in part by the increased expression and/or secretion of MIF in the cancer cell and tumor microenvironment.

Tumor-associated macrophages (TAMs) and tumor-infiltrating lymphocytes (TILs) contribute significantly to the development of immunosuppressive properties of a tumor. TAMs show a number of pro-tumorigenic features. Macrophages may display a broad spectrum of phenotypes where type 1(M1 ) and type 2 (M2) macrophages represent its extremes. M1 stimulate inflammation, produce proinflammatory cytokines, and show anti-tumor cytotoxic activity while M2 produce anti-inflammatory cytokines, extracellular matrix components, and remodeling enzymes and show high phagocytic and low cytotoxic activities. TAMs support tumor progression by producing pro-angiogenic and growth factors. They are also thought to inhibit T cell effector functions by releasing immunosuppressive cytokines. In most of studied cancers, the presence of increased number of TAMs appears to be a marker of poor prognosis. Tumor-infiltrating lymphocytes (TILs) represent another important part of tumor stromal cells. They are found in different tumors, and their population is mainly comprised of CD3+ and CD8+ T cells. CD3+ T cells have antitumor activity. As TAMs, CD3+ TILs show both antitumor and tumor-supporting activities. In contrast, CD8+ T lymphocytes have cytotoxic activity against cancer cells, and these T cells could play an important role in antitumor immunity. Regulatory T cells (Tregs) also show immunosuppressive activity in cancer. In a healthy organism, Tregs control activation and expansion of B and T cells, as well as NK cell cytotoxicity; however, in cancer, they inhibit antitumor immune responses. Interestingly, Tregs may act differently at different stages of tumor development. At the initial stages, Tregs suppress inflammation that may lead to carcinogenesis but later diminish antitumor immunity via the secretion of immunosuppressive cytokines and inhibition of cytotoxic cell function.

“MIF-induced cancer cell immunosuppressed phenotype” as used herein refers to the effect MIF has on tumor infiltrated immune cells, and how it participates in the induction of an immunosuppressed phenotype of the cancer cells, which are no detected and/or attacked by immune cells. The methods described herein, relying on the administration of MIF targeting agent aim at reversing the immunosuppressed phenotype for example by enhancing the polarization of pro-tumorigenic M2 macrophages, inhibiting the infiltration of immune suppressive cells into the tumor microenvironment and/or enhancing the infiltration of antitumor immune cells infiltration into the tumor microenvironment.

In one aspect, the MIF targeting agent is selected from the group consisting of anti-MIF antibody, anti-MIF isoform antibody, anti-oxidized MIF antibody, anti-MIF receptor antibody, MIF receptor inhibitor, CD74 inhibitor, MIF blocking peptide, CAR-T cell, anti-MIF antibody drug conjugate and anti-MIF small molecule inhibitor.

In another aspect, the method further includes administering to the subject a further therapeutic treatment. In some aspects, the treatment is selected from the group consisting of immune checkpoint inhibitor therapy, chemotherapy and radiotherapy.

In one aspect, the alteration in the KEAP1/NRF2 pathway is selected from the group consisting of a KEAP1 gene mutation, NFE2L2/NRF2 mutation, genomic loss of KEAP1, reduced KEAP1 protein expression, increased NRF2 protein expression, and/or protein and/or gene expression modification in a gene or protein in the KEAP1/NRF2 pathway. In another aspect, the cancer cells further include a KRAS mutation and/or a STK11/LKB1 alteration.

In one embodiment, the invention provides a method of identifying a subject as a responder to a macrophage migration inhibitory factor (MIF) targeting agent therapy including identifying an alteration in a KEAP1/NRF2 pathway in a cancer cell in the subject, wherein the presence of the alteration is indicative of a subject who is a responder, and the absence of the alternation is indicative of a subject who is a non-responder, thereby identifying a subject as a responder to a MIF targeting agent therapy.

The methods described herein aim at treating cancer in subject in need thereof by targeting MIF in cancer cells, thereby instating or reinstating sensitivity of the cancer cells to checkpoint inhibitor, in order to induce a strong and efficient anti-cancer cell immune response in the subject. As discussed, the present invention relies on the discovery that targeting MIF is an efficient strategy, in patients having a tumor presenting an alteration in a KEAP1/NRF2 pathway, as such alteration is responsible for increased MIF expression and secretion. The methods described herein are initially described for targeting NSCLC but can be applied to any cancer presenting an alteration in a KEAP1/NRF2 pathway.

Cancer is a group of diseases involving abnormal cell growth with the potential to invade or spread to other parts of the body. In 2015, about 90.5 million people had cancer, about 14.1 million new cases occur a year and it caused about 8.8 million deaths (15.7% of deaths). The most common types of cancer in males are lung cancer, prostate cancer, colorectal cancer and stomach cancer. In females, the most common types are breast cancer, colorectal cancer, lung cancer and cervical cancer. The term “cancer” refers to a group of diseases characterized by abnormal and uncontrolled cell proliferation starting at one site (primary site) with the potential to invade and to spread to other sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof. As used herein, “neoplasm” or “tumor” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, rectal, liver, kidney, lung, testicular, breast, ovarian, pancreatic, brain, and head and neck cancers, melanoma, sarcoma, multiple myeloma, leukemia, lymphoma, and the like.

Exemplary cancers described by the national cancer institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms'Tumor.

In one aspect, the subject has non-small cell lung cancer (NSCLC).

By “identifying a subject as a responder to a macrophage migration inhibitory factor (MIF) targeting agent therapy”, it is meant that the methods rely on the identification of the subject that are likely to benefit from a MIF targeting agent therapy (i.e., those that are likely responder), versus the subject that are not likely to benefit from a MIF targeting agent therapy. With MIF targeting agent therapy aiming at reducing MIF level, MIF secretion, and/or MIF signaling, identifying a subject as a responder to a MIF targeting agent” include identifying subject having increased expression levels and/or secretion levels of MIF. This includes identifying subject having an alteration in a KEAP1/NRF2 pathway in a cancer cell, which is responsible for increased MIF expression and/or MIF secretion.

An increased expression and/or secretion of MIF (e.g., as indicated by an alteration in a KEAP1/NRF2 pathway) is likely indicative of a subject that will benefit from a treatment with a MIF targeting agent therapy. Such patient should therefore be treated with a MIF targeting agent therapy. However, an absence of an increased expression and/or secretion of MIF (e.g., as indicated by the absence of an alteration in a KEAP1/NRF2 pathway) is likely indicative of a subject that will not benefit from a treatment with a MIF targeting agent therapy. Such patient should therefore not be treated with a MIF targeting agent therapy.

MIF expression and/or secretion and the alterations in the KEAP1/NRF2 pathway are biomarker that can be used for the identification of the patients that are likely to benefit from a MIF targeting agent therapy, as well as for the identification of patients that are not likely to benefit from a MIF targeting agent therapy.

In one aspect, the alteration in the KEAP1/NRF2 pathway is selected from the group consisting of a KEAP1 gene mutation, NFE2L2/NRF2 mutation, genomic loss of KEAP12133, reduced KEAP1 protein expression, increased NRF2 protein expression, and/or protein and/or gene expression modification in a gene or protein in the KEAP1/NRF2 pathway. In another aspect, the cancer cells further comprise a KRAS mutation and/or a STK11/LKB1 alteration.

In one aspect, the method further includes administering to the subject a further therapeutic treatment. In some aspects, the treatment is selected from the group consisting of immune checkpoint inhibitor therapy, chemotherapy and radiotherapy.

Presented below are examples discussing the effect of KEAP1/NRF2 pathway alteration on cancer cell response to MIF inhibition contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### EXAMPLE 1

### Methods

The data details in the following examples (e.g., including response to anti-PD1 treatment) were performed using subcutaneous murine models for in vivo analysis.

Deep profiling and characterization of tumor microenvironment (TME) heterogeneity of KRAS-mutant (K) and LKB1 (KL), and/or KEAP1 deficient (KK and KLK) tumors were performed using single-cell RNA sequencing (scRNA-seq) and multiplex staining.

Data from pre-clinical models has been used to survey the immune genomic data available from the MD Anderson ICON study (a cohort of early-stage lung cancer untreated 148 resected tumors) and TCGA lung cohorts to further validate our findings.

### EXAMPLE 2

### LKB1/STK11 and KEAP1 Alterations are Genomic Determinants of Immunotherapy Resistance

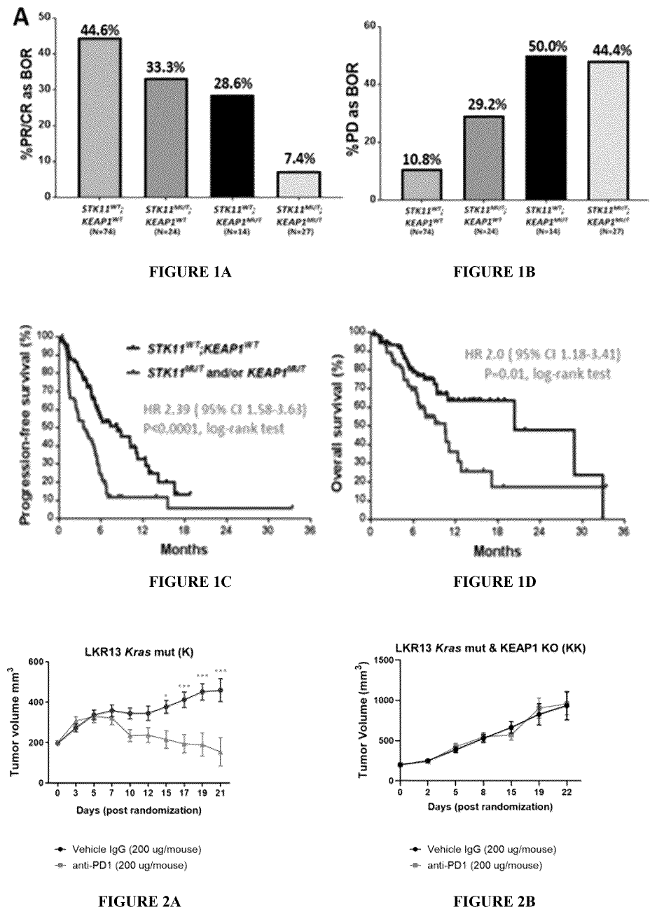

As illustrated in FIGS. 1A-1D, KEAP1-deficient tumors showed reduced response rate to immune checkpoint blockade in both, patients and preclinical models. Clinical data from NSCLC patients (n=139) treated with pembrolizumab plus standard care first-line chemotherapies indicated a higher percentage of patients with refractory diseases associated with STK11/LKB1 and/or KEAP1 mutations (FIGS. 1A and 1B). Furthermore, in patients with STK11 and/or KEAP1 genomic alterations, the survival analysis showed that the addition of pembrolizumab to chemotherapy did not result in clinical benefit compared to chemotherapy alone (FIGS. 1C and 1D).

### EXAMPLE 3

### KEAP1 Knock-Out Tumors Display Lack of Response to Anti-PD-1 Treatment In Vivo

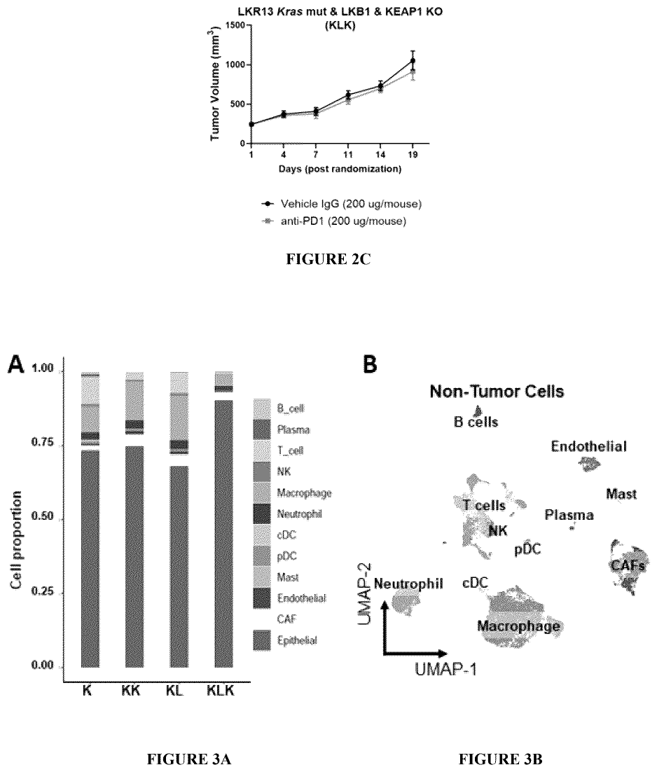

In order to better understand the specific contribution of KEAP1 loss in immunotherapy resistance, the response of tumors with or without KEAP1 knockout (KO) to anti-PD1 treatment was first tested. LKR13 Kras-mutant (K), LKR13 Kras-mutant and KEAP1 KO (KK), and LKR13 Kras-mutant and KEAP1 and LKB1 KO (KLK) tumor cells were subcutaneously injected into the flank of immunocompetent 129/sv mice. Once tumors reached 150-250 mm3, animals were randomized to receive anti-PD-1 or IgG control. As illustrated in FIGS. 2A-2C, while K tumors showed significant response to anti-PD1 treatment, KEAP1 loss alone or with additional loss of LKB1 completely impaired therapeutic response to anti-PD1 treatment.

### EXAMPLE 4

### KEAP1 Loss Modulates Tumor Immune Infiltration

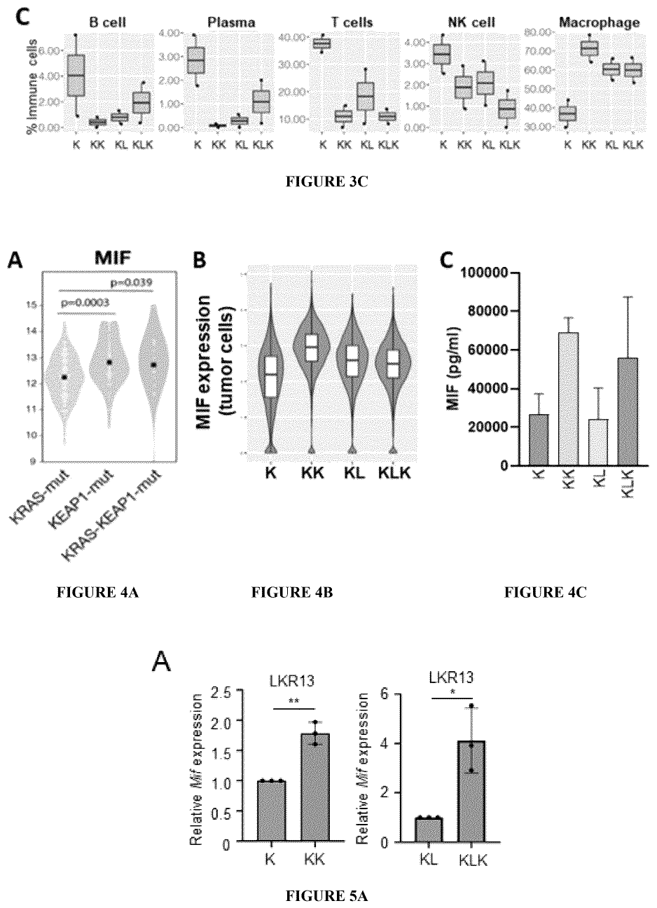

To investigate the impact of KEAP1 on tumor immune infiltration, single cell RNA sequencing (scRNA-seq) analysis on tumor samples derived from syngeneic Kras-mutant tumor models (K) with depletion of LKB1 (KL), KEAP1 (KK) or both LKB1 and KEAP1 (KLK) were performed. As illustrated in FIGS. 3A-3C, KEAP1 deficient tumors displayed a notable reduction in tumor immune infiltration with a reduction of T and B cells and increased M2 polarization.

### EXAMPLE 5

### KEAP1-Deficient Tumors Upregulate MIF Expression and Secretion

An analysis of TCGA lung cancer cohort revealed that MIF is upregulated in KK tumors compared to KRAS mutant (K) lung adenocarcinomas (LUADs) (FIG. 4A). Likewise, LKR13 KK and KLK mouse tumor models displayed increased MIF expression from scRNA-seq analysis (FIG. 4B) and MIF secretion in vitro (FIG. 4C). These results suggested that MIF, a known regulator of macrophage polarization and immune response, may contribute to KEAP1-loss mediated immunotherapy resistance.

To validate the findings from the scRNA-seq analysis, qRT-PCR were performed on LKR13 cell lines. Specifically, both LKR13KK and KLK demonstrated significantly elevated MIF mRNA levels in comparison to LKR13K and KL, respectively (FIG. 5A). In parallel, there was a marked increase in MIF protein abundance in LKR13KK and KLK relative to LKR13K and KL, respectively (FIG. 5B). To further validate this relationship, wild-type KEAP1 was overexpressed in human cell lines H2030, H23, and H2122, all of which possess mutant KEAP1. Post-overexpression, a pronounced reduction in protein abundance was evident in all three cell lines (FIG. 5C). This underscores that KEAP1 negatively regulates MIF expression.

### EXAMPLE 6

### MIF Depletion Reduces Cell Proliferation and Increases PD-L1 Expression in KEAP1-Deficient Tumor Cells

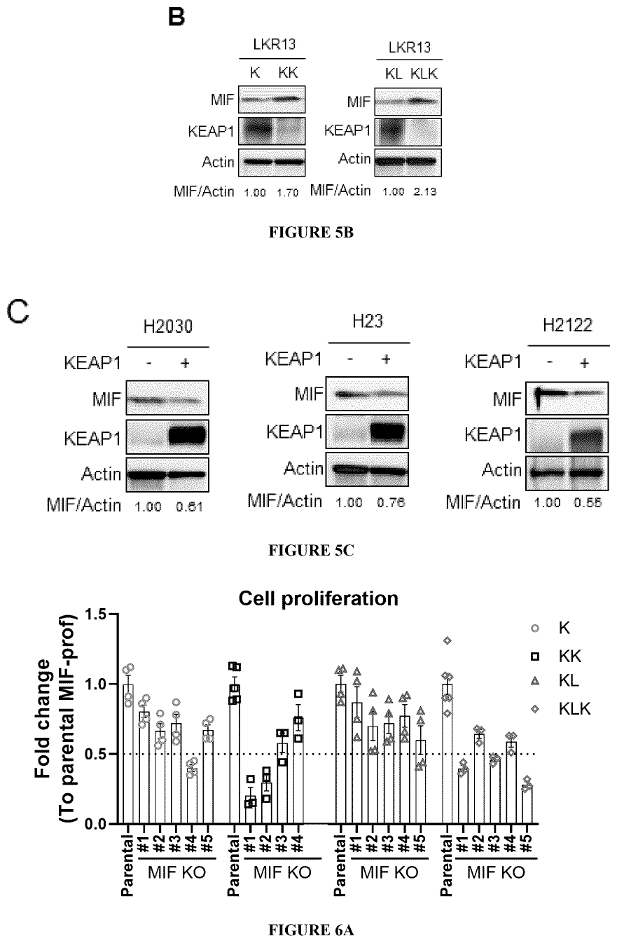

To investigate the impact of MIF expression in KEAP1 and/or LKB1-deficient cells, MIF knock-out (KO) tumor cells were generated by using CRISPR-Cas9 technology in Kras-mutant LKR13 K, KL, KK and KLK murine models. The data indicated that MIF loss is associated with a robust reduced cell proliferation rate in tumor cells lacking KEAP1 expression (FIG. 6A). Additionally, depletion of MIF increased PD-L1 expression in vitro (FIG. 6B).

### EXAMPLE 7

### MIF Loss Does Not Significantly Reduce K and KL Tumor Growth but Significantly Reduces KK and KLK Tumor Growth In Vivo

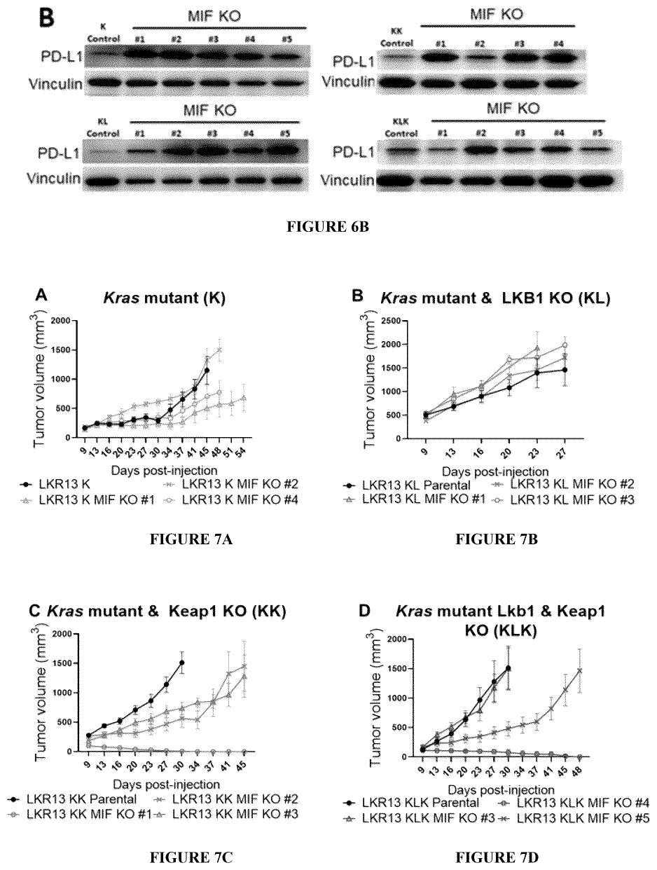

To determine the role of MIF in mediating the growth of KEAP1 deficient tumors in vivo, K, KL, KK and KLK cells with or without MIF expression were injected into the flank of immunocompetent 129/Sv mice and tumor growth rate of MIF-KO (vs. MIF-wt) tumors were evaluated. MIF depletion did not significantly reduce K (FIG. 7A) and KL (FIG. 7B) tumor growth but significantly impaired the growth of KK (FIG. 7C) and KLK (FIG. 7D) tumors in vivo. These data provide further evidence supporting that MIF plays an important role in the growth of KEAP1 deficient tumors.

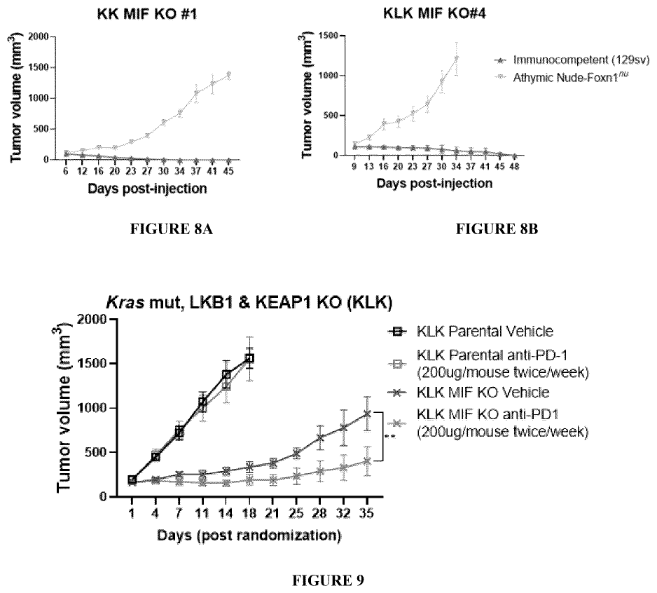

Further, to evaluate whether tumor growth impairment associated to MIF depletion was T cells dependent KK and KLK MIF KO clones that underwent through a total rejection when injected in immune competent mice were injected in immunodeficient mice. KK MIF KO #1 and KLK MIF KO #4 were injected in the flack of immune athymic Nude mice lacking T and NK cell immune populations (FIGS. 8A and 8B). Tumor lacking MIF expression did not show a reduced tumor growth rate when injected in immune deficient mice, indicating that T and NK cell populations may play a critical role in the tumor growth impairment associated with MIF loss.

### EXAMPLE 8

### MIF Loss Overcomes Immunotherapy Resistance in KLK Tumors

To evaluate the specific role of MIF in mediating immunotherapy resistance in KEAP1 deficient tumors, KLK MIF-KO and-wt cells were injected in the flank of immunocompetent 129/Sv mice. When tumors reached a volume of 150 mm³, mice were randomized to vehicle or treatment with immune checkpoint blockade (anti-PD1). As expected, KLK tumors showed a complete lack of response to anti-PD1 immunotherapy. Loss of MIF significantly reduced tumor growth and treatment with anti-PD1 further impaired tumor growth in KLK models (FIG. 9). Collectively, these data demonstrate that blockade of MIF is a promising therapeutic strategy to increase anti-tumor immune response of checkpoint inhibition therapy in patients harboring KEAP1-deficient NSCLC tumors.

### EXAMPLE 9

### Conclusion

While K tumors showed significant response to anti-PD1 treatment, KEAP1 loss completely impaired therapeutic response to this immunotherapy. KEAP1-deficient tumors were characterized by low immune infiltration while displayed an enrichment of cancer associated fibroblasts (CAFs) and endothelial cells. scRNA-seq data indicated a significant reduction of T cell infiltration, in particularly, CD8 and NK T cells, pronounced decreased of B cell population and a marked M2 macrophages polarization.

Concordantly, KEAP1-deficient pre-clinical tumors showed a significant upregulation of MIF expression and secretion, and CRISPR-Cas9 deletion of MIF dramatically impaired in vivo tumor growth in KK and KLK but not in K or KL models.

These findings indicate that loss of KEAP1, alone or in combination with STK11/LKB1 alterations, unfavorably reprograms tumor microenvironment. These changes appear to be mediated at least in part through MIF upregulation, providing a potential therapeutic strategy for overcoming KEAP1 loss-dependent resistance to immunotherapy.

Although the invention has been described with reference to the above examples, it will be understood that modifications and variations are encompassed within the spirit and scope of the invention. Accordingly, the invention is limited only by the following claims.

1. A method of treating non-small cell lung cancer (NSCLC) in a subject comprising:
a) identifying an alteration in a KEAP1/NRF2 pathway in a cancer cell in the subject, and
b) administering to the subject identified with a KEAP1/NRF2 pathway alteration, an anti-macrophage migration inhibitory factor (MIF) agent, thereby treating NSCLC in the subject.

2. The method of claim 1, wherein identifying an alteration in a KEAP1/NRF2 pathway comprises detecting a KEAP1 gene mutation, NFE2L2 /NRF2 mutation, genomic loss of KEAP1, reduced KEAP1 protein expression, increased NRF2 protein expression, and/or protein and/or gene expression modification in a gene or protein in the KEAP1/NRF2 pathway in the cancer cell.

3. The method of claim 1, wherein the cancer cell further comprises a KRAS mutation and/or a STK11/LKB1 alteration.

4. The method of claim 1, wherein the MIF targeting agent is selected from the group consisting of an anti-MIF antibody, anti-MIF isoform antibody, anti-oxidized MIF antibody, anti-MIF receptor antibody, MIF receptor inhibitor, CD74 inhibitor, MIF blocking peptide, CAR-T cell, anti-MIF antibody drug conjugate and anti-MIF small molecule inhibitor.

5. The method of claim 4, comprising administering to the subject a further therapeutic treatment selected from the group consisting of immune checkpoint inhibitor therapy, chemotherapy and radiotherapy.

6. (canceled)

7. The method of claim 5, wherein the immune checkpoint inhibitor therapy comprises a PD-1 inhibitor and/or a PD-L1 inhibitor.

8. The method of claim 7, wherein the PD-1 inhibitor and/or a PD-L1 inhibitor are selected from the group consisting of nivolumab, pembrolizumab, atezolizumab, avelumab, durvalumab, cemiplimab, and dostarlimab.

9. The method of claim 1, wherein treating NSCLC comprises inhibiting NSCLC cell proliferation, reducing NSCLC tumor growth and/or increasing NSCLC patient survival.

10. A method of reducing immune checkpoint therapy resistance, increasing immune checkpoint therapy sensitivity and/or increasing anti-tumor activity in a subject having cancer comprising administering to the subject an anti-macrophage migration inhibitory factor (MIF) agent,
wherein the subject's cancer cells have a KEAP1/NRF2 alteration,
thereby reducing immune checkpoint therapy resistance, increasing immune checkpoint therapy sensitivity and/or increasing anti-tumor activity in the subject.

11. The method of claim 10, wherein the subject has non-small cell lung cancer (NSCLC).

12. The method of claim 10, wherein reducing immune checkpoint therapy resistance and/or increasing immune checkpoint therapy sensitivity comprises increasing cancer cell PD-L1 expression, decreasing MIF level and/or MIF secretion, and/or increasing anti-tumor activity.

13-14. (canceled)

15. The method of claim 12, wherein increasing anti-tumor activity comprises reversing MIF-induced cancer cell immunosuppressed phenotype, increasing cancer cell sensitivity to immune checkpoint blockade, inhibiting tumor-associated immune evasion, increasing tumor immune infiltration and/or inhibiting pro-tumorigenic M2 macrophage polarization.

16. The method of claim 10, wherein the MIF targeting agent is selected from the group consisting of anti-MIF antibody, anti-MIF isoform antibody, anti-oxidized MIF antibody, anti-MIF receptor antibody, MIF receptor inhibitor, CD74 inhibitor, MIF blocking peptide, CAR-T cell, anti-MIF antibody drug conjugate and anti-MIF small molecule inhibitor.

17. The method of claim 10, comprising administering to the subject a further therapeutic treatment selected from the group consisting of immune checkpoint inhibitor therapy, chemotherapy and radiotherapy.

18. (canceled)

19. The method of claim 10, wherein the alteration in the KEAP1/NRF2 pathway is selected from the group consisting of a KEAP1 gene mutation, NFE2L2/NRF2 mutation, genomic loss of KEAP1, reduced KEAP1 protein expression, increased NRF2 protein expression, and/or protein and/or gene expression modification in a gene or protein in the KEAP1/NRF2 pathway.

20. The method of claim 10, wherein the cancer cells further comprise a KRAS mutation and/or a STK11/LKB1 alteration.

21. A method of identifying a subject as a responder to a macrophage migration inhibitory factor (MIF) targeting agent therapy comprising identifying an alteration in a KEAP1/NRF2 pathway in a cancer cell in the subject,
wherein the presence of the alteration is indicative of a subject who is a responder, and the absence of the alternation is indicative of a subject who is a non-responder,
thereby identifying a subject as a responder to a MIF targeting agent therapy.

22. The method of claim 21, wherein the alteration in the KEAP1/NRF2 pathway is selected from the group consisting of a KEAP1 gene mutation, NFE2L2/NRF2 mutation, genomic loss of KEAP1, reduced KEAP1 protein expression, increased NRF2 protein expression, and/or protein and/or gene expression modification in a gene or protein in the KEAP1/NRF2 pathway.

23. The method of claim 21, wherein the cancer cells further comprise a KRAS mutation and/or a STK11/LKB1 alteration.

24. The method of claim 21, further comprising administering to the subject a further therapeutic treatment selected from the group consisting of immune checkpoint inhibitor therapy, chemotherapy and radiotherapy.

25. (canceled)
